(19) United States
(12) Patent Application Publication
(10) Pub. No.: US 2025/0381352 A1
(43) Pub. Date: Dec. 18, 2025

(54) Overdose Reviver Pressurized Spray

(71) Applicant: Keith Alan Roberts, Melbourne, FL (US)

(72) Inventor: Keith Alan Roberts, Melbourne, FL (US)

(21) Appl. No.: 18/741,050

(22) Filed: Jun. 12, 2024

(51) Int. Cl. A61M 15/08; A61M 11/00

(52) U.S. Cl. CPC A61M 15/08; A61M 11/006

(57) ABSTRACT

The Overdose Reviver Pressurized Spray, provides two different methods to administer Narcan solution intranasally, containing enough Narcan solution, and pressurized air for at least ten one second 4 mg squirts of Narcan solution with or without a 3″ long extension air tube. To aim from the base of a nostril, depress an atomizing air release valve, administering atomized, pressurized Narcan solution into the nostrils of an unconscious, opioid overdosed person. Or to remove an air release valve, insert a pressurized air canister with Narcan solution into The Overdose Reviver Automatic Inhaler, supplying the necessary pressurized air, and Narcan solution for an Automatic Inhaler to perform its automatic reviving method. Wherein a microprocessor chip detects a life-threatening low heart rate, an Automatic Inhaler will automatically, repeatedly, administer atomized, pressurized Narcan solution into the nostrils of an unconscious opioid overdosed person once every two to three minutes and alert for help.

### BACKGROUND OF THE INVENTION

Narcan is an opioid overdose reversal agent administered intranasally. Wherein opioids, and especially fentanyl overdose deaths is an out-of-control epidemic. The U.S.

is being saturated with fentanyl. Last year in 2023 the U.S. recorded over 100,000 opioid overdose deaths, wherein over 60,000 people have been revived with Narcan. Wherein a currently available Narcan nasal administering method is a one-time use, single dose, disposable dispenser. Wherein USJP4789089B2 Illustrates a dispenser housing style using a thumb depressing plunger. Wherein US2023/0321367A1 is a similar style dispenser using reloadable drug vials. However neither contain the necessary composition or volume to achieve a desired output. Wherein U.S. Pat. No. 8,802,058B2
is an aerosol drug disperser wherein the composition takes the form of a form.

Whereas if someone needs more than one dose of Narcan administered, and only one Narcan dispenser is on scene, wherein it would be time consuming, and a desperate scramble to acquire more Narcan wherein a life could be rapidly slipping away.

The Overdose Reviver Pressurized Spray is a small pressurized air canister containing Narcan solution in the form of a liquid, wherein a responder can aim from the base of someone's nostrils, whereby depressing said atomizing air release valve on said pressurized air canister, said responder can administer atomized, pressurized Narcan solution into a nostril. Wherein too much Narcan presents no problem, Whereas zero Narcan could be fatal. Wherein Narcan will not affect a person without opioids in their system.

### SUMMARY OF THE INVENTION

Wherein using a currently available Narcan nasal administering dispenser, wherein said responder needs to be able to reach the floor, wherein physically insert said Narcan nasal dispenser into a nostril of an unconscious, opioid overdosed person. Wherein using said dispenser requires two fingertips and a thumb, wherein depressing said plunger releases Narcan solution.

Wherein if a person falls unconscious said person could land in an awkward position, between a bed and a wall making it difficult for said responder to move, or maneuver dead weight. Wherein said responder could have physical limitations, strength, arthritis, or be confined to a walker, or a wheelchair. Wherein using said pressurized air canister with an extension spray tube, said responder can aim from the base of someone's nostril, whereby depressing said atomizing air release valve administers atomized, pressurized Narcan solution into a nostril of an unconscious opioid overdosed person.

Wherein said pressurized air canister provides two different methods of administering atomized, pressurized Narcan solution intranasally. Wherein to use as described, or remove said air release valve, insert said pressurized air canister into The Overdose Reviver Automatic Inhaler, whereby supplying the necessary composition and volume wherein said Automatic Inhaler can perform its automatic reviving method, wherein said Automatic Inhaler has an air release valve permanently disposed within the inside.

The Overdose Reviver Automatic Inhaler is a method to administer Narcan solution intranasally, and automatically. Wherein said Automatic Inhaler is worn strapped to a user's neck, wherein a nasal airline with nasal prongs, is strapped and secured in place under said user's nose with head, and nasal airline elastic straps. Wherein said Automatic Inhaler is battery powered, mechanical, and automated. Wherein a microprocessor chip receives data from heartbeat sensors, whereby detecting a life-threatening low heart rate said microprocessor chip, activates the electrical and mechanical programmed functions, wherein administering atomized, pressurized Narcan solution to said user's nostrils once every two to three minutes and alert for help. Wherein said Automatic Inhaler is intended for someone who knowingly is using opioids, most likely home alone, and wants to take a precautionary preemptive action to automatically revive themself in case a lethal dose is ingested. Wherein the application number for The Overdose Reviver Automatic Inhaler is Ser. No. 18/739,862.

### BRIEF DESCRIPTION OF THE DRAWINGS

FIG. 1 Is a perspective view of the preferred embodiment, a pressurized air canister with an air release valve, and Narcan solution.

FIG. 2 Is a perspective view of an embodiment, a currently available Narcan solution nasal administering dispenser.

FIG. 3 Is a sectional view of an embodiment, a silhouette, the profile of an adult face, forehead, nose, lips and chin.

FIG. 4 Is a cutaway side view of an embodiment, The Overdose Reviver Automatic Inhaler and inner components.

FIG. 5 Is a side view of an embodiment, The Overdose Reviver Automatic Inhaler and a nasal airline with nasal prongs is strapped, and secured to a profile of an adult face.

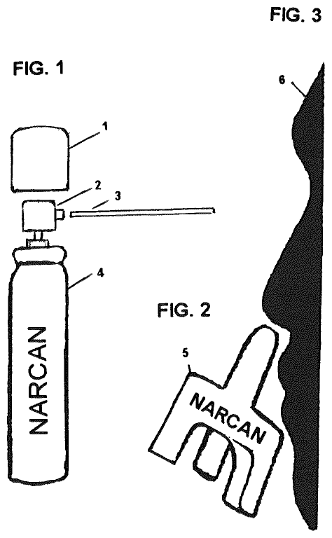

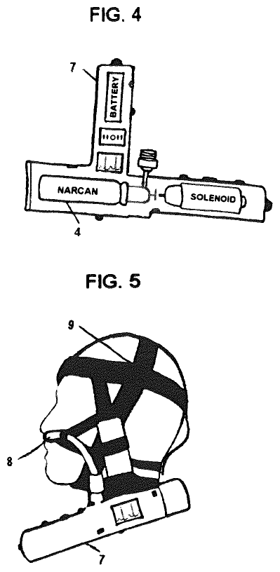

### DETAILED DESCRIPTION OF THE PREFERRED EMBODIMENTS

The Overdose Reviver Pressurized Spray is described herein as “Pressurized Spray” consisting of; a valve covering top cap 1, an atomizing air release valve 2, a 3″ long extension air tube 3, a pressurized air canister containing Narcan solution 4, wherein said Pressurized Spray contains enough Narcan solution, and pressurized air for at least ten one second 4 mg squirts of Narcan solution. Wherein said pressurized air canister is tubular shaped, wherein the dimensions are approximately ⅞″ width by 4″ length.

The Pressurized Spray provides two different methods of administer Narcan solution intranasally. Method one wherein a responder can use as is, with or without an extension air tube 3, wherein said responder can aim from the base of nostrils, depress said atomizing air release valve, administer atomized, pressurized Narcan solution into nostrils of an unconscious, opioid overdosed person. Method two is to remove said air release valve 2, wherein insert said pressurized air canister 4 into The Overdose Reviver Automatic Inhaler 7. Wherein supplying the necessary pressurized air, and Narcan solution for The Overdose Reviver Automatic Inhaler to perform its reviving method. FIG. 2 is an embodiment of a currently available Narcan solution nasal administering dispenser. FIG. 3 is a sectional view, a silhouette, a profile of an adult face, forehead, nose, lips and chin. FIG. 2 and FIG. 3 is simulating the placement of said currently available Narcan solution nasal administering dispenser.

Wherein said Automatic Inhaler 7 is strapped to a user's neck, wherein a nasal airline with nasal prongs 8, is secured and held in place under said user's nose via head, and nasal airline elastic straps with adjustable snaps.

What is claimed:

1. The Overdose Reviver Pressurized Spray provides two different methods of administering Narcan solution intranasally.

2. Wherein to use said Pressurized Spray as is with or without an extension air tube to aim from the base of nostrils depress an atomizing air release valve administering atomized pressurized Narcan solution into the nostrils of an unconscious opioid overdosed person.

3. Wherein to remove said air release valve on said pressurized air canister containing Narcan solution whereby inserting said pressurized air canister into The Overdose Reviver
Automatic Inhaler supplying the necessary pressurized air and Narcan solution for said Automatic Inhaler to automatically administer atomized pressurized Narcan solution into the nostrils of an opioid overdosed person every two to three minutes and alert for help.
